(19) United States
(12) Patent Application Publication
(10) Pub. No.: US 2024/0074984 A1
(43) Pub. Date: Mar. 7, 2024

(54) ENCAPSULATION OF LIVE MICROORGANISMS FOR GASTROINTESTINALTARGETED DELIVERY

(71) Applicant: Nanyang Technological University, Singapore (SG)

(72) Inventors: Say Chye Joachim LOO, Singapore (SG); Li Ling TAN, Singapore (SG); Manish MAHOTRA, Singapore (SG)

(21) Appl. No.: 18/261,504

(22) PCT Filed: Feb. 18, 2022
(86) PCT No.: PCT/SG2022/050076

§ 371 Date: Jul. 14, 2023

(30) Foreign Application Priority Data
Feb. 19, 2021 (SG) 10202101688R

(51) Int. Cl. A61K 9/50; A61K 35/747

(52) U.S. Cl. CPC A61K 9/5089; A61K 9/5036; A61K 35/747; A61K 2035/128

(57) ABSTRACT

The present invention relates to a method of forming polymeric microparticles housing live microorganisms, the method using the steps of (a) providing a microbial solution comprising a crosslinkable polymeric material compatible with live microorganisms; a protective agent; a microbial population; and water; and a crosslinking agent solution comprising a crosslinking agent and water; and (b) subjecting the microbial and crosslinking agent solutions to spray-drying using a co-axial nozzle configured to spray at least three fluids independently, thereby producing microparticles of a crosslinked polymeric material housing live microorganisms where the microbial solution and crosslinking agent solution are each sprayed through one of the innermost two channels of the co-axial nozzle and an atomizing gas is sprayed through the outermost channel. In a specific embodiment, the polymeric material is alginate, and the microbial population is selected from one or more of probiotic bacteria and/or a live biotherapeutic.

### FIELD OF INVENTION

This invention relates to a method of producing crosslinked dried microparticles containing encapsulated live microorganisms via a facile, single-step spray-drying process, wherein polymer crosslinking occurs at the tip of the spray-dryer nozzle, when the polymer and crosslinker in the inner and outer channels respectively, or vice versa, come into contact when ejected. In particular, this microparticle delivery system (crosslinked dried microparticles) is designed to retain high viability of encapsulated microorganisms even amidst the high temperature conditions transient in the spray-dryer system, and this is achieved via incorporation of protective agents. Further, this delivery system achieves the effect of protecting the encapsulated acid-labile microbes from the acidic pH in the human stomach via a localized buffering mechanism, thereby fulfilling targeted delivery to the human intestine.

### BACKGROUND

The listing or discussion of a prior-published document in this specification should not necessarily be taken as an acknowledgement that the document is part of the state of the art or is common general knowledge.

The human body is home to trillions of microorganisms, most of which reside in the human gut. Over the past few decades, research has shed light on the intricate relationships which exist between these microorganisms, termed the gut microbiota, and human health. The human gut microbiota plays an important role in regulating many aspects of human health, including digestive, immunity and neurological-related aspects. As such, numerous therapeutic and prophylactic interventions have been devised to modify and enhance the gut microbial community. Of which, the use of live microorganisms has garnered increasing attention. These beneficial live microorganisms can be broadly categorized into two types—probiotics and live biotherapeutic products (LBP). Probiotics and LBPs are similar in that they are “viable microorganisms which when administered in adequate amounts confer a health benefit on the host” (Probiotics in Food, Food and Agriculture Organization of the United Nations & World Health Organization, 2006). Most probiotics and LBPs tend to be bacteria from the phyla Firmicutes, such as Lactobacillus, Streptococcus and Bacillus, while Bifidobacterium of the Actinobacteria phyla and Saccharomyces, which are a type of fungi, have also been used. The main differentiation between probiotics and LBPs lies in their regulatory status, where probiotics tend to be loosely regulated as dietary supplements, whereas LBPs are specifically developed as drugs and are regulated by medical regulatory authorities such as the Food and Drug Administration (FDA) and the European Directorate for the Quality of Medicines and healthcare (EDQM) (M. Cordaillat-Simmons, A. Rouanet & B. Pot, Exp. Mol. Med. 2020, 52, 1397-1406).

An important criterion for probiotics and LBPs is that these live microorganisms should retain viability until they reach the target site of action—the human small and large intestine. This involves maintaining high viability counts during (1) the period of shelf storage and (2) passage through the upper gastrointestinal tract (GIT) during oral ingestion. In the shelf storage phase, environmental factors including humidity, ambient heat and oxidative stress can greatly reduce viability of microorganisms. Generally, the expected shelf life of live microorganism formulations, such as in the form of capsules, tablets and sachet, varies between 12-18 months from the date of manufacture, and it is important that in this period, microorganism viability counts in the product are at least the colony forming unit (CFU) dosage as claimed on the package. In the ingestion phase, microorganisms need to first survive the harsh conditions in the upper GIT, before they can reach and colonize the small/large intestine. The gastric environment is considered most hostile for microorganisms, as acidic and enzymatic stomach juices (pH 1.2-3, contains proteases, and half gastric emptying time around 80.5 min (M. T. Cook et al., J. Control. Release 2012, 162, 56-67)) readily kill off most living organisms. In the duodenum, microorganisms are exposed to bile salts from the gall bladder. Bile functions as a biological detergent that emulsifies and solubilizes lipids, hence it has the ability to disrupt the phospholipids of microbial cell membranes (M. Begley, C. G. M. Gahan & C. Hill, FEMS Microbiol. Rev. 2005, 29, 625-651).

These challenges in live microorganism administration hence inspire the development of certain encapsulation formulations to mitigate the antagonistic factors. Of which, encapsulation formulations based on the spray-drying technique have been explored. Spray-drying is a high-throughput, easily scaled-up method of powder production which has been commonly employed in the industrial setting. Via the combined action of atomization, heat and vacuum drying, spray-drying generates dried powder particles rapidly in a single-step, making spray-drying a highly efficient and economical technique. However, in the context of live microorganism encapsulation, the high temperature and rapid dehydration process in spray-drying can greatly reduce bacteria viability. To maintain high microorganism viability during the spray-drying process as well as confer additional functional benefits such as shelf-life stability and gastroprotective properties, polymers are commonly added as an excipient material within the microbial formulation. These polymers are also known as encapsulation materials, and the polymers may be added directly alongside the microorganism, or the polymers may be crosslinked to immobilize the microorganism within the polymeric matrix. Prior-reported live microorganism formulations used encapsulation polymers include alginate, chitosan, gums (arabic, guar, locust bean, xanthan, glucomannan), proteins, carrageenan, pectin, cellulose and starch (WO2014006261; Y. Liu et al., Adv. Funct. Mater. 2020, 30, 2001157; WO2009070012A1; F. B. Haffner, R. Diab & A. Pasc, AIMS Mater. Sci. 2016, 3, 114-136; and G. Broeckx et al., Int. J. Pharm. 2016, 505, 303-318).

Among the variety of encapsulation materials, alginate, a negatively charged linear polysaccharide consisting of 1→4 linked β-(D)-guluronic and α-(L)-mannuronic acids derived from brown algae or bacterial sources, is particularly preferred. The advantages of alginate include that it is biocompatible, generally recognized as safe (GRAS), of natural origin and low cost. Crosslinking of alginate can be achieved by exposure to multivalent cation crosslinkers, such as Ca²⁺, Sr²⁺, Ba²⁺, Zn²⁺ and Al³⁺, which bind readily to the guluronic acid blocks in alginate, thereby forming a gel (Ý. A. Mørch et a., Biomacromolecules 2006, 7, 1471-1480). This crosslinking process is mild, non-deleterious, rapid, and temperature-independent, making it suitable for the immobilization of live microorganisms within. These crosslinked alginate hydrogels are also known to be insoluble in acid and thus, they can confer gastric acid-protective effects on entrapped microorganisms, facilitating their passage into the lower GIT.

Several alginate-based formulations for live microorganism encapsulation have been devised. However, prior formulations mostly focused on utilizing fabrication techniques such as extrusion (U.S. Pat. No. 5,472,648A; and M. Gu et al., Food Hydrocoll. 2019, 91, 283-289), emulsification (C. Yus et al., Polymers 2019, 11, 1668) or layer-by-layer encapsulation (WO2017105990A1; and A. C. Anselmo et al., Adv. Mater. 2016, 28, 9486-9490). Resultant particles formed via these techniques are in the “wet” state, and an additional drying step, such as air drying, freeze-drying, fluidized bed drying or vacuum foam drying, is typically required to yield a final dry product. A milling or micronisation step is also frequently involved following the drying step to yield a homogenous, fine powder product. Comparing these techniques with spray-drying, the major advantage which spray-drying offers is the ability to produce dry fine powder particles directly in a single step, which greatly streamlines and simplifies the formulation process. Having a single-step process could also minimize contamination risks and transfer losses, thereby ensuring higher quality and better yield of the resultant product.

The challenge of using spray-drying for alginate-encapsulated microbial formulations is however, regarding the method of crosslinking alginate in a spray-dryer set-up. Since the spray-drying method directly produces dried powder, it is not feasible to expose the alginate and microorganism slurry to an aqueous crosslinking bath, as in the typical extrusion or emulsification method. It is also counter-productive to first spray-dry an alginate-microorganism slurry, followed by crosslinking in an aqueous multivalent cation salt solution, as this would require an additional step following to dry the formulation. Crosslinking of alginate before feeding into the spray-dryer inlet would also not be feasible as the viscosity of the crosslinked gel would be too high for the feed to be sprayed through the spray-dryer nozzle, causing instances of nozzle clogging. A method of spray-drying crosslinked alginate particles has been described by Zicari et al. (U.S. Pat. No. 9,700,519B2; S. A. Strobel et al., Food Hydrocoll. 2016, 58, 141-149; and S. A. Strobel et al., Ind. Biotechnol. 2018, 14, 138-147). However, this method involves the use of a volatile base to facilitate the crosslinking process, which is highly deleterious to many pH-sensitive probiotic microorganisms, such as lactic acid bacteria. By far, no methods for spray-drying with in situ polymeric crosslinking suitable for the encapsulation of live microorganisms has been described.

Therefore, there is a need to develop a highly scalable and facile technique, through the use of spray-drying, for the encapsulation of live microorganisms with improved viability.

### SUMMARY OF INVENTION

Aspects and embodiments of the current invention will now be discussed by reference to the following numbered clauses.

1. A method of forming polymeric microparticles housing live microorganisms, the method comprising the steps of:
(a) providing:
   a microbial solution comprising
      a crosslinkable polymeric material compatible with live microorganisms;
      a protective agent;
      a microbial population; and
      water; and
   a crosslinking agent solution comprising a crosslinking agent and water; and
(b) subjecting the microbial and crosslinking agent solutions to spray-drying using a co-axial nozzle configured to spray at least three fluids independently, thereby producing microparticles of a crosslinked polymeric material housing live microorganisms, wherein:
   the microbial solution is sprayed as a first feed through an inner channel, the crosslinking agent solution is sprayed as a second feed through an outer channel that surrounds the inner channel; and an atomizing gas is sprayed through a jacketing channel that surrounds the outer channel; or
   the crosslinking agent solution is sprayed as a first feed through an inner channel, the microbial solution is sprayed as a second feed through an outer channel that surrounds the inner channel; and an atomizing gas is sprayed through a jacketing channel that surrounds the outer channel.
      2. The method according to Clause 1, wherein the crosslinkable polymeric material compatible with live microorganisms is selected from one or more of the group consisting of an oligosaccharide, dextrin, or more particularly, alginate, a gum (e.g. Arabic, guar, locust bean, xanthan, glucomannan), a non-bioactive protein (e.g. gelatin, collagen, whey protein, soy protein, or caseinate), carrageenan, pectin, cellulose, and starch.
      3. The method according to Clause 2, wherein the crosslinkable polymeric material compatible with live microorganisms is alginate, optionally wherein the alginate has a guluronic:mannuronic ratio of from 90:10 to 10:90, such as from 70:30 to 30:70.
      4. The method according to any one of the preceding clauses, wherein the crosslinkable polymeric material compatible with live microorganisms has a concentration of from 2 to 10% w/v, such as 2% w/v in the microbial solution.
      5. The method according to Clause 4, wherein the crosslinkable polymeric material compatible with live microorganisms has a viscosity at room temperature of from 5 mPas to 250 mPas (measured at a shear rate of 100/s).
      6. The method according to any one of the preceding clauses, wherein the protective agent is selected from one or more of the group consisting of a sugar (e.g. a monosaccharide, a disaccharide), an amino acid, a protein, a prebiotic, and an antioxidant.
      7. The method according to Clause 6, wherein the protective agent is selected from one or more of the group consisting of caseinate or, more particularly, sucrose, trehalose, lactose, cysteine, methionine, lysine, proline, arginine, skimmed milk, inulin, a fructooligosaccharide, a galactooligosaccharide, ascorbic acid, β-carotene, and a tocopherol.
      8. The method according to any one of the preceding clauses, wherein the protective agent has a concentration of from 5 to 30% w/v, such as from 7 to 25% w/v, such as from 10 to 15% w/v in the microbial solution.
      9. The method according to any one of the preceding clauses, wherein the dry weight percentage of the crosslinkable polymeric material compatible with live microorganisms is from 10 to 90% of the total dry weight, such as about 50%.
      10. The method according to any one of the preceding clauses, wherein the microbial population is selected from one or more of probiotic bacteria and/or a live biotherapeutic, optionally wherein the microbial population is formed from one or more bacteria selected from the genera Saccharomyces, Escherichia, Arkkemansia, Bacteroides, Faecalibacterium, Leuconostoc, or more particularly, Bacillus, lactobacilli (such as one or more of the group consisting of Lactobacillus, Lacticaseibacillus, Lactiplantibacillus, Lentilactobacillus, Levilactobacillus, Ligilactobacillus, Limosilactobacillus, Liquorilactobacillus, Ligilactobacillus, and Companilactobacillus), Bifidobacterium, Streptococcus, and Enterococcus, optionally wherein the probiotic bacteria are non-spore forming bacteria.
      11. The method according to Clause 10, wherein the microbial population is formed from one or more of the group consisting of Lacticaseibacillus rhamnosus, Lacticaseibacillus paracasei, and Lactiplantibacillus plantarum.
      12. The method according to any one of the preceding clauses, wherein the concentration of the microbial population in the microbial solution is from 1*10⁶ CFU/mL to 1*10¹¹ CFU/mL, such as about 3*10⁸ CFU/mL.
      13. The method according to any one of the preceding clauses, wherein the crosslinking agent is selected from one or more of the group consisting of an inorganic salt of a multivalent cation, a polyanion, and genipin.
      14. The method according to Clause 13, wherein the crosslinking agent is selected from one or more of a calcium salt, a magnesium salt, a strontium salt, an aluminium salt, a barium salt, and a zinc salt, optionally wherein the salts are a phosphate, a carbonate, a hydroxide or, more particularly, a chloride or lactate salt.
      15. The method according to Clause 14, wherein the crosslinking agent is calcium chloride.
      16. The method according to any one of the preceding clauses, wherein the concentration of the crosslinking agent solution is from 1 to 100 mM, such as from 5 to 50 mM, such as about 10 mM.
      17. The method according to any one of the preceding clauses, wherein an inlet temperature of a drying gas used in the method is greater than or equal to 100° C., such as from 100 to 150° C., such as about 120° C.
      18. The method according to Clause 17, wherein the microparticles of the crosslinked polymeric material housing live microorganisms are collected on a collection apparatus, wherein the distance between the collection apparatus and the co-axial nozzle, the flow rates of the first and second feed, and the drying gas temperature at the co-axial nozzle are selected to provide a temperature of 60 to 80° C. for the microparticles collected by the collection apparatus.
      19. The method according to any one of the preceding clauses, wherein:
when the first feed is the microbial solution and the second feed is the crosslinking agent solution, then the ratio of the first feed flow rate through the inner channel to the second feed flow rate through the outer channel is from 1:1 to 0.1:1, such as from 1:2 to 1:5; or
when the first feed is the crosslinking agent solution and the second feed is the microbial solution, then the ratio of the first feed flow rate through the inner channel to the second feed flow rate through the outer channel is from 10:1 to 1:1, such as from 2:1 to 5:1.
   20. The method according to Clause 19, wherein:
when the first feed is the microbial solution and the second feed is the crosslinking agent solution, then the flow rate for the first feed is from 1.5 to 2 mL/min and the flow rate for the second feed is from 3 to 4 mL/min, optionally wherein the first feed has a flow rate of about 1.5 mL/min and the second feed has a flow rate of about 3 mL/min; or
when the first feed is the crosslinking agent solution and the second feed is the microbial solution, then the flow rate for the first feed is from 3 to 4 mL/min and the flow rate of the second feed is from 1.5 to 2 mL/min, optionally wherein the first feed has a flow rate of about 3 mL/min and the second feed has a flow rate of about 1.5 mL/min.
   21. The method according to any one of Clause 17 and Clauses 18 to 20, as dependent upon Clause 17, wherein the atomizing gas has a flow rate of from 500 to 1,000 L/hour, such as about 742 L/h.
   22. The method according to any one of the preceding clauses, wherein the co-axial nozzle is operated in one or both of:
(ai) a pneumatic atomization mode; and
(aii) a co-current mode.
   23. The method according to any one of the preceding clauses, wherein the live microorganisms in the microparticles of the crosslinked polymeric material housing live microorganisms remain alive for a period of from 12 to 18 months from the formation of said microparticles.
   24. The method according to any one of the preceding clauses, wherein the microparticles of the crosslinked polymeric material housing live microorganisms have a particle size of 2 to 15 μm.
   25. The method according to any one of the preceding clauses, wherein the microbial solution further comprises a non-microbial bioactive.
   26. The method according to Clause 25, wherein the non-microbial bioactive is selected from one or more of the group consisting of nutraceuticals (e.g. phytochemicals), lipids, and bioactive proteins.
   27. The method according to Clause 26, wherein the non-microbial bioactive is selected from one or more of the group consisting of curcumin, beta-carotene, bovine serum albumin, lysozyme, fish oil, and flaxseed oil, where the oils are dissolved or emulsified.
   28. The method according to any one of the preceding clauses, wherein the crosslinking agent solution further comprises chitosan, so as to provide chitosan-coated microparticles.

### DRAWINGS

FIG. 1 depicts the physical properties of spray-dried crosslinked Ca-ALG-SUC particles with embedded Lacticaseibacillus rhamnosus GG (LGG) bacteria. (a) Photograph of spray-dried Ca-ALG-SUC LGG powder; (b) Scanning electron microscopy (SEM) image of spray-dried Ca-ALG-SUC LGG powder showed round particles in the size range of 2-15 μm; and when the Ca-ALG-SUC LGG powder was rehydrated and washed in 0.9% NaCl saline, the epifluorescence microscopy image in (c) showed LGG bacteria embedded within a calcium-alginate matrix, indicating that effective crosslinking was attained.

FIG. 2 depicts the survivability data of LGG in various formulations post-spray-drying. (a) log₁₀(CFU/g) data of LGG in various formulations following spray-drying; and (b) Change in log₁₀(CFU/g) counts of LGG in various formulations, as compared to before spray-drying. Letters on bars based on * p<0.05, ** p<0.01, *** p<0.001, **** p<0.0001 (one-way ANOVA and post-hoc Tukey test, n=3). Data are expressed as mean with standard deviation bars.

FIG. 3 depicts the survivability data of LGG in various spray-dried formulations after exposure to simulated gastric fluid (SGF). (a) log₁₀(CFU/g) data of LGG in various spray-dried formulations following exposure to SGF; (b) Change in log₁₀(CFU/g) counts of LGG in various formulations after SGF exposure, as compared to before SGF exposure (freshly spray-dried microorganism powder); and (c) log₁₀(CFU/g) data of LGG in SUC formulation, exposed to SGF of various pH. Letters on bars based on * p<0.05, ** p<0.01, *** p<0.001, **** p<0.0001 (one-way ANOVA and post-hoc Tukey test, n=3). Data are expressed as mean with standard deviation bars. B.d. indicates that the cell count is below the detection limit, which is approximately 1.5 log₁₀(CFU/g) for the drop plating method used.

FIG. 4 depicts that the localized pH buffering effect contributes to enhanced survivability of LGG in spray-dried crosslinked polymeric formulations. (a) log₁₀(CFU/g) data of LGG in un-crosslinked (ALG) and crosslinked (Ca-ALG-SUC) formulations exposed to SGF in varying powder:SGF ratios; (b) Change in log₁₀(CFU/g) counts of LGG, as compared to before SGF exposure; and (c) pH of SGF media following addition of Ca-ALG-SUC and ALG powder. Data are expressed as mean of triplicates with standard deviation bars.

FIG. 5 depicts that the encapsulated LGG retains high viability and gastroprotective effects following storage. log₁₀(CFU/g) survivability data of LGG in the Ca-ALG-SUC matrix, when the spray-dried powder was freshly prepared, or after the powder was stored for (a) 4 weeks; and (b) 8 weeks in 4° C. refrigerated conditions. Data are expressed as mean of triplicates with standard deviation bars.

FIG. 6 depicts that crosslinking agent on the outer feed channel provides better gastroprotective function on encapsulated microorganisms. log₁₀(CFU/g) survivability data of LGG in two formulations—With crosslinking agent in the outer feed channel and polymeric-microorganism slurry in inner feed channel, or vice versa. Data are expressed as mean with standard deviation bars.

FIG. 7 depicts the survivability data of Lactiplantibacillus plantarum (L. plantarum) and Lacticaseibacillus paracasei (L. paracasei) in the crosslinked spray-dried polymeric formulation post-spray-drying. (a) log₁₀(CFU/g) data of L. plantarum and L. paracasei in the Ca-ALG-SUC formulation following spray-drying; and (b) Change in log₁₀(CFU/g) counts of L. plantarum and L. paracasei in the Ca-ALG-SUC formulation, as compared to before spray-drying. Data are expressed as mean with standard deviation bars.

FIG. 8 depicts the survivability data of L. plantarum and L. paracasei in the crosslinked spray-dried polymeric formulation after exposure to SGF. (a) log₁₀(CFU/g) data of L. plantarum and L. paracasei in the Ca-ALG-SUC formulation following exposure to SGF; (b) Change in log₁₀(CFU/g) counts of L. plantarum and L. paracasei in the Ca-ALG-SUC formulation after SGF exposure, as compared to before SGF exposure (freshly spray-dried microorganism powder); (c) log₁₀(CFU/g) data of L. plantarum in SUC formulation, exposed to SGF of various pH; and (d) log₁₀(CFU/g) data of L. paracasei in SUC formulation, exposed to SGF of various pH. Data are expressed as mean with standard deviation bars. B.d. indicates that the cell count is below the detection limit, which is approximately 1.5 log₁₀(CFU/g) for the drop plating method used.

FIG. 9 depicts the survivability data of LGG in various spray-dried formulations after shelf-storage. (a) log₁₀(CFU/g) data of LGG in freshly spray-dried Ca-Alg-Suc-Tre, Ca-Alg-Suc-Tre-Cys and Ca-Alg-Suc-Tre-Asc formulations following exposure to SGF/simulated intestinal fluid (SIF); (b) log(CFU/g) data of LGG in various formulations after 8 weeks, room temperature storage; and (c) log(CFU/g) data of LGG in various formulations after 8 weeks, refrigerated storage. Data are expressed as mean of triplicates with standard deviation bars.

### DESCRIPTION

It has been surprisingly found that the spray-drying method disclosed herein provides a highly scalable and facile technique for the encapsulation of live microorganisms with improved viability compared to conventional techniques. This method advantageously solves the problems associated with the prior fabrication techniques as described hereinbefore, delivering a dried live microorganism powder product, imbued with desirable key attributes, such as high microorganism viability counts, targeted delivery of live microorganisms to the small/large intestine and good product stability throughout the shelf-life duration. Thus, in a first aspect of the invention, there is provided a method of forming polymeric microparticles housing live microorganisms, the method comprising the steps of:
(a) providing:
   a microbial solution comprising
      a crosslinkable polymeric material compatible with live microorganisms;
      a protective agent;
      a microbial population; and
      water; and
   a crosslinking agent solution comprising a crosslinking agent and water; and
(b) subjecting the microbial and crosslinking agent solutions to spray-drying using a co-axial nozzle configured to spray at least three fluids independently, thereby producing microparticles of a crosslinked polymeric material housing live microorganisms, wherein:
   the microbial solution is sprayed as a first feed through an inner channel, the crosslinking agent solution is sprayed as a second feed through an outer channel that surrounds the inner channel; and an atomizing gas is sprayed through a jacketing channel that surrounds the outer channel; or
   the crosslinking agent solution is sprayed as a first feed through an inner channel, the microbial solution is sprayed as a second feed through an outer channel that surrounds the inner channel; and an atomizing gas is sprayed through a jacketing channel that surrounds the outer channel.

It is believed that using a co-axial nozzle design for partitioning a polymer and a crosslinking agent, to achieve in situ crosslinking during the spray-drying process is not something previously done before. Furthermore, the resultant formulation is also imbued with gastroprotective properties, able to maintain high viability of encapsulated microorganisms exposed to bile salts and, more particularly, gastric pH.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As will be appreciated, the spray-drying process reduces the amount of moisture in the resulting polymeric microparticles housing live microorganisms. This may be any suitable level of “dryness”. For example, the polymeric microparticles housing live microorganisms may have a residual moisture level of less than or equal to 5% w/w, such as less than or equal to 4% w/w, such as less than 4% w/w.

An essential part of the invention is the use of a co-axial nozzle that is configured to spray at least three fluids independently. As will be appreciated, the co-axial nozzle is required to provide at least three fluid channels, though the nozzle may have four, five or six channels etc. In a co-axial nozzle having three fluid channels, these are arranged concentrically around a central axis, with the innermost channel (corresponding to the “inner” channel above) being surrounded by the middle channel (corresponding to the “outer” channel above), which is in turn surrounded by the outermost channel (corresponding to the “jacketing” channel above). In a co-axial nozzle having three fluid channels, the jacketing channel supplies the atomizing gas, and atomizes the feeds provided through the inner and outer channels. In one arrangement, the inner channel may provide the microbial solution feed, while the outer channel provides the crosslinking agent solution feed. In another arrangement, the inner channel may provide the crosslinking agent solution feed, while the outer channel provides the microbial solution feed. As will be appreciated, if there are four or more channels, then the outermost channel remains as the jacketing channel, with the remaining channels providing various feeds, which must contain the microbial solution feed and the crosslinking agent solution feed, with other feed potentially providing additional components and/or excipients. Any suitable arrangement of these feeds is possible and contemplated herein.

As noted above, the microbial solution comprises a crosslinkable polymeric material compatible with live microorganisms, a protective agent, a microbial population and water.

The microbial population may be formed from any suitable microorganism or mixtures thereof. For example, the microbial population may be selected from one or more of non-spore forming bacteria and/or a live biotherapeutic. As used herein, the term “live biotherapeutic” refers to a live microbial organism that is efficacious in the prevention, treatment or cure of a disease or condition, and is not a vaccine. In particular embodiments that may be mentioned herein, the microbial population may be formed from one or more bacteria selected from the genera Saccharomyces, Escherichia, Arkkemansia, Bacteroides, Faecalibacterium, Leuconostoc, or more particularly, Bacillus, lactobacilli (such as one or more of the group consisting of Lactobacillus, Lacticaseibacillus, Lactiplantibacillus, Lentilactobacillus, Levilactobacillus, Ligilactobacillus, Limosilactobacillus, Liquorilactobacillus, Ligilactobacillus, and Companilactobacillus), Bifidobacterium, Streptococcus, and Enterococcus. For example, the microbial population may be formed from one or more of the group consisting of Lacticaseibacillus rhamnosus, Lacticaseibacillus paracasei, and Lactiplantibacillus plantarum. In certain embodiments, the probiotic bacteria are non-spore forming.

Any suitable concentration of the microbial population in the microbial solution may be used, provided that a sufficient amount of the microbial population ends up in the polymeric microparticles to be efficacious. In embodiments of the invention that may be mentioned herein, the concentration of the microbial population in the microbial solution may be from 1*10⁶ CFU/mL to 1*10¹¹ CFU/mL, such as about 3*10⁸ CFU/mL.

When used herein, the term “crosslinkable polymeric material compatible with live microorganisms” refers to a polymeric material that can be crosslinked by a crosslinking agent and which does not adversely affect the viability of the microorganisms that form the microbial population, both in the microbial solution (as the free polymeric material) and as the crosslinked polymeric material (following spray-drying). In embodiments of the invention that may be mentioned herein, the viability of the microorganisms that form the microbial population may be from about 8 to 11 log(CFU/g), such as from 9 to 10.5 log(CFU/g), such as from 9.5 to 10 log(CFU/g). Examples of suitable crosslinkable polymeric material compatible with live microorganisms include, but are not limited to, an oligosaccharide, dextrin, or more particularly, alginate, a gum (e.g. Arabic, guar, locust bean, xanthan, glucomannan), a non-bioactive protein (e.g. gelatin, collagen, whey protein, soy protein, or caseinate), carrageenan, pectin, cellulose, starch, and combinations thereof.

In particular embodiments that may be mentioned herein, the crosslinkable polymeric material compatible with live microorganisms may be alginate. The alginate may have any suitable guluronic:mannuronic ratio. For example, alginates used in certain embodiments of the invention may have a guluronic:mannuronic ratio of from 90:10 to 10:90, such as from 70:30 to 30:70.

It is noted that the mechanism for alginate in situ crosslinking via spray-drying is challenging as it takes place rapidly and may have a tendency to clog the nozzles if the parameters are not accordingly optimized. However, the skilled person following the teachings provided herein can select parameters that generate the desired product without the clogging issue becoming problematic.

Any suitable concentration of the crosslinkable polymeric material compatible with live microorganisms may be used in the method disclosed herein, provided that it is sufficient to entrap and immobilize the microorganisms. In particular embodiments of the invention that may be mentioned herein, the crosslinkable polymeric material compatible with live microorganisms may have a concentration of from 2 to 10% w/v, such as 2% w/v in the microbial solution. Correspondingly, the dry weight percentage of the crosslinkable polymeric material compatible with live microorganisms may be from 10 to 90% of the total dry weight, such as about 50%. When used herein, the term total dry weight refers to the weight of the non-aqueous components of the microbial solution and the crosslinking agent solution. That is, the dry microorganisms, the crosslinking agent, the protective agent, and the crosslinkable polymeric material. As will be appreciated, the mass typically attributable to the bacteria (when dried) and the crosslinking agent may be relatively small and negligible when compared to the mass of the protective agent and the crosslinkable polymeric material.

As will be appreciated, the crosslinkable polymeric material compatible with live microorganisms may be a viscous material at room temperature. Any suitable viscosity level may be used in the method disclosed. For example, the crosslinkable polymeric material compatible with live microorganisms may have a viscosity at room temperature of from 5 mPas to 250 mPas (measured at a shear rate of 100/s).

When used herein, the term “protective agent” refers to an agent that allows for the following functional attributes.
AA) Maintain high viability, i.e. <0.2 log CFU reduction, following spray-drying.
AB) Provide good microorganism stability throughout stipulated shelf-life of between 12 to 18 months.
AC) Serve protective function for a variety of microbial strains, such as from the genera described above (e.g. Bifidobacterium, Bacillus, Lactobacillus, Streptococcus, and Enterococcus).
AD) Is of food-grade status.
AE) Should not interfere with the polymeric crosslinking process.
AF) Provide good powder characteristics following spray-drying.

Any suitable material may be used as a protective agent, provided that it meets the requirements above. Examples of general classes of material that can be used as a protective agent include, but are not limited to, a sugar (e.g. a monosaccharide, a disaccharide), an amino acid, a protein, a prebiotic, an antioxidant and combinations thereof. Specific examples of protective agents include, but are not limited to, caseinate or, more particularly, sucrose, trehalose, lactose, cysteine, methionine, lysine, proline, arginine, skimmed milk, inulin, a fructooligosaccharide, a galactooligosaccharide, ascorbic acid, β-carotene, a tocopherol, and combinations thereof. It will be appreciated that milk (e.g. skimmed milk) is a mixture comprising lactose and proteins, amongst other components and so may be used as a protective agent herein.

The protective agent may be used in any suitable amount, provided that it achieves the effects AA to AF above. For example, the total concentration of the protective agent(s) in the microbial solution may be from 5 to 30% w/v, such as from 7 to 25% w/v, such as from 10 to 15% w/v.

As will be appreciated, any material that physically crosslinks the crosslinkable polymeric material compatible with live microorganisms may be used as a crosslinking agent. This may be by hydrogen or, more particularly, ionic or covalent bonding. Examples of crosslinking agents that may be mentioned herein include, but are not limited to, an inorganic salt of a multivalent cation, a polyanion, genipin, and combinations thereof. Specific crosslinking agents that may be mentioned herein include, but are not limited to, a calcium salt, a magnesium salt, a strontium salt, an aluminium salt, a barium salt, a zinc salt, and combinations thereof. The counterion to the cations may be selected from one or more of a phosphate, a carbonate, a hydroxide or, more particularly, a chloride or lactate salt. In particular embodiments that may be mentioned herein, the crosslinking agent may be calcium chloride.

The crosslinking agent may be used in any suitable amount in the method disclosed herein, provided that it is used in a sufficient quantity to generate the desirable effects discussed herein. For example, the crosslinking agent solution may have a concentration of from 1 to 100 mM, such as from 5 to 50 mM, such as about 10 mM. For the avoidance of doubt, the concentration referred to relates to the concentration of the crosslinking agent in the crosslinking agent solution.

To achieve effective in situ crosslinking via spray-drying requires a non-trivial design, involving optimization of parameters including: crosslinker type, crosslinker concentration, polymer concentration, inlet temperature, feed flow rate, flow rate ratio of inner and outer channels, and gas flow rate. For instance, at excessively high crosslinker concentration, clogging of the spray-dryer nozzle can result, while too low concentration of crosslinker fails to achieve effective crosslinking. We now discuss some of the possible suitable parameters that can be used to achieve effective crosslinking, while maintaining high viability of microorganisms and ensuring good powder characteristics. It is also noted that the resultant formulation is also imbued with gastroprotective properties, able to maintain high viability of encapsulated microorganisms exposed to gastric pH and intestinal bile salts. It is noted that the skilled person provided with the general principles disclosed herein would be able to optimize conditions to arrive at a desired product.

As part of a spray-drying process, there may be two gasses used. The first is the atomizing gas, which is used to produce droplets of the feed(s). The second gas is a drying gas, which is typically used to induce rapid drying of the droplets produced by the atomizing gas. Any suitable temperature of the drying gas that enables rapid drying of the droplets, while retaining their activity may be used herein. For example, a suitable temperature for the drying gas that may be used herein may be greater than or equal to 100° C., such as from 100 to 150° C., such as about 120° C. This temperature may be the inlet temperature. For the avoidance of doubt, the “inlet temperature” refers to the heated drying gas temperature, measured right before its entry into the drying chamber of the spray-drying apparatus.

The microparticles of the crosslinked polymeric material housing live microorganisms may be collected on a collection apparatus. In such embodiments, the distance between the collection apparatus and the co-axial nozzle, the flow rates of the first and second feed, and the drying gas temperature at the co-axial nozzle may be selected to provide a temperature of from 60 to 80° C. for the microparticles collected by the collection apparatus. As will be appreciated, the exact parameters used will depend on the spray-drying apparatus being used and may be selected by the skilled person to achieve the desired temperature range.

In certain embodiments that may be disclosed herein, when the first feed is the microbial solution and the second feed is the crosslinking agent solution, then the ratio of the first feed flow rate through the inner channel to the second feed flow rate through the outer channel may be from 1:1 to 0.1:1, such as from 1:2 to 1:5. Additionally or alternatively, when the first feed is the microbial solution and the second feed is the crosslinking agent solution, then the flow rate for the first feed may be from 1.5 to 2 mL/min and the flow rate for the second feed may be from 3 to 4 mL/min (e.g. the first feed may have a flow rate of about 1.5 mL/min and the second feed may have a flow rate of about 3 mL/min). It is noted that these feed rates may help to achieve a temperature of from 60 to 80° C. for the microparticles collected at the collection apparatus.

In alternative embodiments that may be disclosed herein, when the first feed is the crosslinking agent solution and the second feed is the microbial solution, then the ratio of the first feed flow rate through the inner channel to the second feed flow rate through the outer channel may be from 10:1 to 1:1, such as from 2:1 to 5:1. Additionally or alternatively, when the first feed is the crosslinking agent solution and the second feed is the microbial solution, then the flow rate for the first feed may be from 3 to 4 mL/min and the flow rate for the second feed may be from 1.5 to 2 mL/min (e.g. the first feed may have a flow rate of about 3 mL/min and the second feed may have a flow rate of about 1.5 mL/min). It is noted that these feed rates may help to achieve a temperature of from 60 to 80° C. for the microparticles collected at the collection apparatus.

In embodiments of the invention seeking to achieve a temperature of from 60 to 80° C. for the microparticles collected at the collection apparatus, the atomizing gas may be selected to have a suitable flow rate. For example, the atomizing gas may have a flow rate of from 500 to 1,000 L/hour, such as about 742 L/h.

The drying gas flow rate may be controlled by an aspirator. Any suitable aspirator rate may be used in the spray-dryer for the drying gas. For example, the aspirator rate may be from 20 m³/h to 35 m³/h, such as 35 m³/h. These aspirator rates may be useful in obtaining a temperature of from 60 to 80° C. for the microparticles collected at the collection apparatus.

It will be appreciated that multiple parameters may be adjusted by the skilled person in line with the teachings provided herein to achieve the desired temperature of from 60 to 80° C. for the microparticles collected at the collection apparatus.

In the methods disclosed herein, the nozzle of the spray-dryer may be operated in any suitable mode. For example, the co-axial nozzle may be operated in one or both (e.g. one) of:
(ai) a pneumatic atomization mode; and
(aii) a co-current mode.

As noted hereinbefore, the method disclosed herein provides polymeric microparticles housing live microorganisms. It is desirable that the microorganisms remain alive for an extended period of time. Thus, in embodiments of the invention that may be mentioned herein, the live microorganisms in the microparticles of the crosslinked polymeric material housing live microorganisms remain alive for a period of from 10 to 24 months, such as from 12 to 18 months from the formation of said microparticles.

The method may produce any suitable size of microparticle, which is defined herein as a particle having a particle size of less than 50 μm. More particularly, the microparticles of the crosslinked polymeric material housing live microorganisms may have a particle size of from 2 to 15 μm.

In certain embodiments that may be mentioned herein, further active agents may be present in the microparticles made by the method. In embodiments that make use of a co-axial nozzle with more than three channels, the additional channel(s) may each be used to provide a feed containing a non-microbial bioactive. In embodiments that make use of three channels, a non-microbial bioactive may form part of the microbial solution. Any suitable non-microbial bioactive material may be used herein. For example, the non-microbial bioactive may be selected from one or more of the group consisting of nutraceuticals (e.g. phytochemicals), lipids, and bioactive proteins. In particular embodiments that may be mentioned herein, the non-microbial bioactive may include, but is not limited to, curcumin, beta-carotene, bovine serum albumin, lysozyme, fish oil, flaxseed oil, and combinations thereof, where the oils are dissolved or emulsified.

In certain embodiments of the method that is disclosed herein, it may be desirable to form a coating on the microparticles. This may be achieved by adding a coating agent to the crosslinking agent solution (where one uses a co-axial nozzle with only three nozzles) or it may be introduced as a separate feed when the co-axial nozzle has more than three nozzles. A suitable coating agent that may be mentioned in embodiments of the invention disclosed herein is chitosan. The concentration of the coating agent (e.g. chitosan) in any feed that it is present in may be from 0.01 to 1% w/v. For example, in embodiments using a co-axial nozzle having three channels where chitosan is used as the coating agent, the chitosan may be added to the crosslinking agent solution (e.g. the crosslinking agent may be calcium chloride). In this case, from 0.01 to 1% w/v of chitosan may be added to a calcium chloride solution, with the pH adjusted to 6 before use.

Chitosan is not desirable to be used as the crosslinkable polymeric material because chitosan has been known to have an antimicrobial effect. In addition, the acidic pH required to solubilize chitosan can also be deleterious to the viability of pH-sensitive microorganisms. As noted above, in one embodiment of this invention, it is possible to incorporate chitosan on the outer layer of the spray-dried particle, by feeding a chitosan-calcium chloride solution to the outer feed channel of the nozzle. This prevents direct exposure of chitosan to the encapsulated microorganisms in solution, and chitosan forms a layer surrounding the crosslinked alginate particle by an electrostatic mechanism. A chitosan-outer layer can potentially provide improved mucoadhesive properties to the epithelial cells of the small or large intestine, facilitating microbial colonization.

Further aspects and embodiments of the invention may include the following clauses.
BA. Crosslinked dried polymeric microparticles loaded with live microorganisms comprising:
ia) A crosslinked dried polymeric matrix;
ib) Protective agent(s) (e.g., caseinate or, more particularly, sucrose, trehalose, inulin, ascorbic acid, cysteine);
ic) Live microorganisms, such as probiotics or live biotherapeutics, encapsulated within the crosslinked dried polymeric matrix.
BB. A method to synthesize crosslinked dried polymeric microparticles comprising:
iia) dissolution of polymer with protective agent(s);
iib) sterilization of polymeric slurry from step (iia);
iic) mixing live microorganisms with the polymeric slurry from step (iib);
iid) spray-drying using a three-fluid co-axial nozzle, where the polymeric slurry and crosslinking agent are fed separately into the inner and outer channels respectively, or vice versa. Spray-drying parameters, including gas flow rate, inlet temperature and outlet temperature are optimized to preserve bioactivity and good storage properties of the active ingredient, where the fluid flow rate and flow rate ratio (inner relative to outer channel) can also be tuned to control the particle size and degree of crosslinking;
iie) collection of crosslinked spray-dried powder at collecting chamber, for incorporation into dosage forms.

As noted above, the disclosed single-step spray-drying method produces live microorganisms encapsulated in crosslinked polymeric particles. The described method utilizes a spray-drying nozzle that can separately feed at least three fluids (e.g. three fluids), also known as concentric coaxial spray-drying nozzles, for partitioning a polymer and a crosslinking agent in the middle (i.e. outer) and innermost feed channels respectively, or vice versa, while an atomizing gas is fed through the outermost channel. Crosslinking occurs at the tip of the spray-dryer nozzle when both the polymer and crosslinking agent are ejected. The method may make use of alginate polymer combined with a calcium salt crosslinker. However, other crosslinkers, such as strontium, zinc, aluminum salts, or other polymers, such as polysaccharide gums, proteins, carrageenan, pectin, and starch, in combination with suitable crosslinking agents can also be formulated using the described technique.

The resultant product arising from the technique described in this invention, is a dried crosslinked powder with encapsulated live microorganisms (i.e. in the form of polymeric microparticles housing live microorganisms). A resultant dry powder product is favored as it allows for a prolonged shelf-life of the microorganisms. In a dried format, molecular mobility tends to be minimal, hence cellular components of microorganisms are preserved and microorganisms enter a dormant state which allows them to remain stable for a long period of time. Dried formulations are also more versatile and convenient for dosing, as dried powder formulations can be incorporated into capsules, tablets, and sachets. In other embodiments, a liquid dosage format may be used; however, liquid dosage formulations typically utilize spore-forming microbes, such as Bacillus and Saccharomyces, which have inherently higher resistance to environmental stresses. Hence, for non-spore forming microbes, such as Lactobacilli, Bifidobacterium, Streptococcus, Enterococcus, it is generally still regarded that the dry-powder format would provide greatest shelf-stability.

In the described method, a high CFU/g (e.g. >109), dry, encapsulated microorganism powder can be obtained. By optimization of various spray-drying parameters and the incorporation of protective agents as detailed herein, low log CFU reduction can be achieved during the spray-drying process, ensuring maximal CFU/g counts in the final powder formulation.

By way of the mechanism of crosslinking for encapsulation of live microorganisms, the method described herein also provides a gastroprotective function. That is, conferring protective properties to the encapsulated microorganisms during their passage through an acidic gastric fluid media. This characteristic is possible due to a localized buffering effect achieved with a crosslinked polymeric matrix. Excess protons in the vicinity of encapsulated microorganisms are sequestered by the polymer, thereby maintaining a higher pH around the encapsulated microorganisms than in the bulk gastric acid media. This allows for the live microorganism formulation to be consumed even on empty or fasting stomach conditions, where pH levels are lowered to around 2, while still allowing for high quantities of viable microorganisms to reach the lower GIT. Since the crosslinked polymeric matrix itself provides an acid-protective property, there is no need for further use of enteric polymeric capsules or films, like phthalates or acrylates, which are synthetic and often not generally recognized as safe (GRAS).

In one embodiment of this invention involving the use of alginate and a calcium salt crosslinker, the crosslinked alginate matrix also confers protection against protease enzymes in the gastric milieu. Alginate has been shown to inhibit the action of pepsin, the main protease found in the human stomach. In a crosslinked calcium-alginate matrix, microorganisms are released in the small intestine by the action of phosphate ions sequestering the crosslinking agent. Bile acid binding properties have also been observed of alginate, suggesting that the alginate matrix can protect encapsulated microorganisms against bile insults in the duodenum, facilitating their delivery to the lower GIT (Niu et al, Food Chem. 2019, 270, 223-228.). Another benefit of using crosslinked alginate matrix is the higher glass transition temperature conferred by crosslinking. Generally, a glassy phase formed surrounding the microorganism can induce sufficient viscosity to arrest molecular mobility to a minimum, thereby allowing for better preservation of cellular components, thereby yielding a longer shelf-life for these microorganisms.

The method disclosed herein for the formation of polymeric microparticles housing live microorganisms is a facile, single-step and scalable method. In the disclosed method, crosslinking is achieved in situ during the spray-drying process, allowing for simultaneous atomization, crosslinking, and drying of the formulation. Compared to prior techniques used for the encapsulation of live microorganisms, such as extrusion, emulsification or layer-by-layer encapsulation, an additional drying and/or milling step is not required, hence streamlining the fabrication process. Spray-drying is also considerably more scalable than prior techniques, making this method highly suitable for the production of dry encapsulated live microorganism formulations in industrial-scale quantities.

In summary, the method disclosed herein provides the following advantages.
1) Facile, single-step, scalable method of producing dry encapsulated live microorganism formulations.
2) Ability to produce encapsulated, dry forms of non-spore forming bacteria, such as from the genera Lactobacillus, Bifidobacterium, Streptococcus, Enterococcus.
3) Maintain high viability counts of encapsulated microorganism after spray-drying.
4) Gastro-protective effect on encapsulated live microorganisms.
5) Targeted delivery of live microorganisms to the lower GIT, i.e. small and large intestine.
6) Good microorganism stability throughout stipulated shelf-life of between 12-18 months.

Method to Synthesize Crosslinked Dry Polymeric Microparticles:

A selected polymer together with the selected protective agents, may be dissolved in appropriate concentrations in distilled water. The examples of polymers and protective agents which can be used, as well as their respective concentrations, are detailed above and in the subsection—Choice of polymers and composition of protective agents. The crosslinking agent may be prepared separately by dissolving a suitable concentration in distilled water. The types of crosslinking agents and their suitable concentrations are detailed above and in the subsection—Choice of cross/inking agent. Sterilization of the polymeric slurry and crosslinking agent may then be conducted, typically by heat sterilization, involving autoclaving at 121° C. or higher, for a duration of 15 minutes or longer. Sterilization by filtration across a 0.45 μm or finer filter or may also be conducted, but this method involves frequent replacement of filter mesh and also use of high pressure (due to high viscosity of the polymeric slurry), hence may be less cost-efficient than autoclaving.

Live microorganisms are prepared by inoculating in suitable broths and incubating for a stipulated duration. The typical culture medium comprises of a nitrogen source, a carbon source, various growth factors required by the organism, and water. For most Lactobacillus strains, a suitable media is de Man, Rogosa and Sharpe (MRS). Incubation temperatures can range from 30 to 42° C. and incubation durations can range from 16 to 72 hours, depending on the selected species. Stationary phase cultures are typically preferred as maximum cell yield is obtained. Several Lactobacillus strains have also demonstrated highest resistance to drying when harvested in the stationary phase. Microorganism cultures may then be washed by three cycles of centrifugation and replacement with a saline solution, typically 0.9% (w/v) NaCl. Suitable centrifugation conditions may be from 6,000 to 14,000×g (e.g. 6,000 to 12,000×g), for a duration of from 5 to 20 minutes, depending on the volume of culture prepared. After washing, a small amount of saline may be added to resuspend the pellet into a liquid microorganism mixture, before transferring the microorganism mixture to the polymeric slurry and mixing thoroughly.

The spray-dryer may be set up to operate in the co-current mode, with assembly of a three-fluid co-axial nozzle which consists of an inner fluid channel, an outer fluid channel and an atomizing gas channel. In a typical run for encapsulation of microorganisms, the parts of the spray-dryer may be well sterilized with 70% ethanol followed by pre-heating to a temperature of 140° C. or higher, to sterilize the components and dry-out any residual moisture in the apparatus. Once completed, the inlet temperature of the spray-dryer may be set to the pre-determined temperature suitable for spray-drying the microorganism formulation. The selection of suitable spray-drying parameters is detailed above and in the subsection—Choice of spray-drying parameters. The polymer-microorganism slurry and crosslinking agent may then be fed into the inner and outer feed channels, respectively, at preset flow rates and flow rate ratios. Another configuration involves the polymer-microorganism slurry and crosslinking agent being fed in the outer and inner channels respectively instead, however, it has been experimentally determined that the configuration with the polymer-microorganism slurry in the inner channel and the crosslinking agent in the outer channel achieves better protection of encapsulated microorganisms against simulated gastric acid milieu (see the Examples section below). Without wishing to be bound by theory, this may be due to more effective crosslinking achieved when the crosslinking agent is fed through the outer channel, facilitated by the mechanism of solvent evaporation in a spray-dryer. As the inner and outer feeds are ejected from the spray-dryer nozzle, an atomizing gas is simultaneously ejected in the outer-most channel. The atomized droplets get exposed to the heated drying gas in the chamber. This results in quicker evaporation of solvent from the outer feed channel. With the crosslinking agent in the outer channel evaporating first, this results in the crosslinker preferentially mixing with the inner polymeric slurry, facilitating effective crosslinking.

Following spray-drying, the crosslinked spray-dried microorganism powder may be collected at the collecting chamber. An optional post-stabilization step to further remove excess moisture, and/or a sifting step to generate uniformly sized particles, may also be incorporated. The harvested powder may then be mixed with other excipients, such as antacids (e.g. magnesium hydroxide) or, more particularly, whitening agents (e.g., titanium dioxide), anti-caking agents (e.g., magnesium stearate, silicon dioxide), fillers (e.g., maltodextrin), prebiotics (e.g., inulin, fructooligosaccharides, galactooligosaccharides), antioxidant agents (e.g., caseinate or, more particularly, ascorbic acid, cysteine) and/or flavoring, before being incorporated into a final dosage format, such as tablets, capsules, or sachets.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of the polymeric microparticles housing live microorganisms, which confers a therapeutic effect on the treated patient (e.g. sufficient to treat or prevent the disease). The effect may be objective (i.e. measurable by some test or marker) or subjective (i.e. the subject gives an indication of or feels an effect).

The polymeric microparticles housing live microorganisms may be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the polymeric microparticles housing live microorganisms and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of the polymeric microparticles housing live microorganisms in any pharmaceutical formulation used in accordance with the present invention will depend on various factors, such as the severity of the condition to be treated, the particular patient to be treated, as well as the microbial material which is/are employed. In any event, the amount of polymeric microparticles housing live microorganisms in the formulation may be determined routinely by the skilled person.

Herein the polymeric microparticles housing live microorganisms may also be referred to as “active ingredient”.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives. It will be appreciated that parenteral formulations may be reconstituted just before use by the addition of water or saline.

Depending on the disorder, and the patient, to be treated, as well as the route of administration, the polymeric microparticles housing live microorganisms may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 10,000 mg per day of polymeric microparticles housing live microorganisms.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

Choice of Spray-Drying Parameters:

Nozzle: Since the direct mixing of the polymer and the crosslinking agent can lead to formation of a highly viscous gel, commonly used two-channel nozzles are not suitable. Concentric multichannel nozzles which allow simultaneous flow of at least two feed fluids during the spray-drying process are required. For instance, three-fluid nozzles, which include two feed channels, and one atomizing gas channel are suitable. A three-fluid nozzle may use a pneumatic atomization mode where a compressed gas flowing through the nozzle breaks the fluid droplet at the tip of the nozzle into a spray mist.

Gas flow: A stream of compressed air or inert gas like nitrogen can be used as the atomizing and the drying gas. Spray-dryers are usually operated in a co-current mode i.e. drying gas and atomized droplets flowing in the same direction or counter-current mode i.e. drying gas and atomized droplets flowing in the opposite direction. The co-current mode introduces a high temperature drying gas in the nozzle tip, facilitating rapid drying of the solvent. The loss of the thermal energy of the flowing drying gas due to the solvent evaporation process results in a lower temperature at the collector assembly. On the other hand, in a counter-current mode, higher temperatures at the collector are observed as the hot drying gas enters from the collector section. Spray-dryers operating in the co-current mode are preferred as lower temperatures in the collector can improve the survivability of the heat-labile microorganisms.

Gas flow rate: A higher atomizing gas flow rate leads to smaller particle size as there is greater disruption to the feed solution during spraying. A smaller droplet size facilitates a more rapid and efficient drying, therefore, a high atomizing gas flow rate may be used herein. Examples of suitable atomizing gas flow rates are provided hereinbefore.

Inlet and Outlet temperature: In a co-current mode, the inlet temperature refers to the temperature of the drying gas in the nozzle region while the outlet temperature refers to the temperature at the collector assembly. Solvent evaporation occurs at the tip of the nozzle; hence the inlet temperature should be sufficiently high (>100° C. for aqueous feeds) for efficient evaporation. Further drying and stabilization occurs at the drying chamber and the collector assembly, hence the outlet temperature should also be carefully optimized to ensure maximal microorganism viability and good powder characteristics. Outlet temperature is lower than the inlet temperature in a co-current mode, as thermal energy of the drying gas is reduced approaching the collector due to cooling effects from solvent evaporation. The outlet temperature is mainly determined by the inlet temperature and fluid flow rates, i.e. higher the drying gas temperature, higher the outlet temperature; the higher the fluid flow rate, the lower the outlet temperature. A mild outlet temperature range of between 60 to 80° C. (i.e. from 60 to 80° C.) has been found to better preserve viability of microorganisms in the final dried powder. Accordingly, a suitable inlet temperature can be selected to obtain dry powder characteristics and maximum survivability of the microorganisms.

Feed Flow Rates: In a three-fluid nozzle setup, the inner and outer channel feed flow rates play an important role in determining the size as well as the interaction between the fluids at the tip of the nozzle. Higher flow rates allow for a higher-throughput process, while lower flow rates facilitate formation of smaller, more efficiently dried droplets. Hence, an intermediate-to-low flow rate is recommended. Flow rate ratios of the fluids in a multi-channel nozzle can affect the interaction between the fluids and particle morphology. Similarly, the nozzle blockage due to the excessive gelation of the polymer at the tip can be avoided by using a suitable flow rate ratio. Additionally, since flow rates affect the outlet temperature, optimization is done to achieve a suitable outlet temperature for spray-drying of the microorganisms. Suitable flow rates are discussed hereinbefore.

Choice of Polymers and Composition of Protective Agents:

Type of polymer: Various types of polymers may be used, such as but not limited to, alginate, gums (arabic, guar, locust bean, xanthan, glucomannan), proteins, carrageenan, pectin, cellulose and starch. An important criterion in the selection of polymers is that these polymers, together with their suitable solvents, should be compatible and non-deleterious to microorganism. Polymers soluble in an aqueous medium are preferred, as the aqueous solvent is not deleterious to microorganism survival, and water is easily removed via the spray-drying method. Among alginates, various types may be used, such as from different sources (seaweed or bacteria), of different guluronic/mannuronic (G/M) acid ratios or of different viscosities. Different alginates can have different gelling kinetics; for instance, higher G-content alginates associate more strongly with multivalent cations, resulting in a higher degree of crosslinking in high G-content alginates. Alginates with lower viscosities are also generally more preferred, as high viscosity of the feed solution can disturb the spray-drying atomization process. For polymers with inherently high viscosities, higher spray-drying inlet temperature can be utilized to maintain good feed flow.

Concentration of polymer: The concentration of the polymer used may be between 2-10% (w/v), the main consideration being that the concentration should be sufficiently high to confer a gastroprotective effect, yet not too high that viscosity becomes a concern. The concentration of polymer selected should also account for the additional viscosity due to addition of protective agents and microorganisms.

Type(s) of protective agent(s): The purpose of incorporating protective agents is to promote microorganism tolerance during the spray-drying and shelf-storage processes. The suitable type of protectant largely depends on the microorganism; however, there are a few that appear to work well with many species. Caseinate or, more particularly, disaccharides, such as sucrose, trehalose, and lactose, are particularly useful as these enhance desiccation tolerance by stabilizing cellular membranes and proteins. Amino acids, such as cysteine, methionine, lysine, proline, and arginine, also serve a similar function, by stabilizing proteins during the spray-drying process. Other complex protectants, such as skimmed milk, which incorporate disaccharides, amino acids, and other proteins, can also be used. Prebiotics, such as inulin, fructooligosaccharides, and galactooligosaccharides, have also demonstrated protective effects on spray-dried microorganisms, possibly by provision of a glassy matrix surrounding the microorganisms. For obligate anaerobic strains, such as Bifidobacterium, antioxidant agents such as caseinate or, more particularly, ascorbic acid, β-carotene, and tocopherols, may be additionally added.

Concentration of protective agent(s): For disaccharides, concentrations of from 5 to 30% (w/v) may be used for a sufficient protective effect to be exerted. For suitable concentrations of other protective agents, such as amino acids, prebiotics and antioxidants, various suitable recipes are known to people skilled in the art. An important criterion in selecting the concentration of protective agents is that these should not interfere with the polymeric crosslinking process. A general guideline for so is that the dry weight percentage (w/w) attributable to the polymer should not be less than 10% of the total.

Choice of Crosslinking Agent:

Type of crosslinking agent: Depending on the polymer used, suitable crosslinking agents may be selected accordingly. For instance, various multivalent cationic salts, such as chloride and lactate salts of calcium, magnesium, strontium, barium, zinc, aluminum, may be used in combination with anionic polymers like alginate, carrageenan, or pectin. The natural crosslinking agent extracted from gardenia fruit, genipin, can also be utilized with proteins, collagen, and gelatin. Importantly, the type of crosslinking agent chosen should be non-deleterious to microorganism survivability and demonstrate proven safety for oral consumption.

Concentration of crosslinker: The suitable concentration of crosslinking agent depends primarily on the type of crosslinker used, the type of polymer used, as well as the concentration of polymer used. An optimized concentration, that is neither too high (which causes clogging of spray-drying nozzle, hence low powder yield), nor too low (results in ineffective crosslinking) needs to be empirically determined. A general guideline is to utilize the maximum crosslinker concentration that can be afforded, without causing nozzle clogging. If clogging occurs even at a low concentration of crosslinker, the flow rate ratio of the outer to inner feed channels may be increased, to mitigate nozzle clogging.

Further aspects and embodiments of the invention will now be discussed by reference to the following non-limiting examples.

### EXAMPLES

Materials

LFR5/60 alginate was purchased from Protanal®, USA. Lacticaseibacillus rhamnosus GG (LGG) was purchased from Culturelle, USA. Lactiplantibacillus plantarum and Lacticaseibacillus paracasei were strains isolated from fermented food sources. Protanal© LFR5/60 sodium alginate (M/G=30/70, M_{w}=20-60 kDa) was procured from FMC Biopolymer, USA. De Man-Rogosa-Sharpe (MRS) powdered media and Bacto agar were purchased from Thermo Fisher Scientific, USA. MRS broth was prepared according to manufacturer's instructions, by dissolving 52 g of MRS powder in 1 L of deionized (DI) water, and adjusting the solution to pH 6.2 by addition of hydrochloric acid (HCl) and sodium hydroxide (NaOH). MRS agar was prepared by adding 1.5% (w/v) Bacto agar to MRS broth. Chemicals used in this experiment, including sucrose, trehalose, calcium chloride (CaCl₂), calcium lactate, sodium ascorbate, L-cysteine, sodium chloride (NaCl), sodium citrate, hydrochloric acid (HCl), sodium hydroxide (NaOH), porcine pepsin, α-amylase from porcine pancreas, phosphate buffered saline (PBS), ethanol and MiniPax absorbent packets were purchased from Sigma Aldrich, USA. The Live/Dead™ BacLight™ Bacterial Viability Kit was purchased from Thermo Fisher Scientific, USA. Sterilization by autoclaving at 121° C. for 15 min was done for every media, agars, chemical or apparatus prior to use, where necessary.

Analytical Techniques

SEM

Morphologies of the spray-dried powders were observed using a JEOL JSM-6360 Scanning Electron Microscope (SEM) (JEOL Ltd., Japan), with an accelerating voltage of 5 kV in the secondary electron mode. Spray-dried powders were mounted to a stub with carbon tape and coated with 10 nm of conductive gold prior to imaging.

Epifluorescence Microscopy

Viability staining of spray-dried probiotic powder was performed using Live/Dead™ BacLight™ Bacterial Viability Kit. Selected spray-dried samples were rehydrated and washed thrice in 0.9% NaCl to remove any un-crosslinked polymer or probiotics. Each sample was then stained with 500-times diluted SYTO 9 and propidium iodide in 0.9% NaCl. Samples were stained for 15 min in dark conditions then fluorescently imaged using the Axio Observer Z1 Inverted Microscope (Carl Zeiss AG, Germany).

General Procedure for the Preparation of Live Microorganisms

Live microorganisms were prepared by inoculating in suitable broths and incubating for a stipulated duration. The typical culture medium comprises of a nitrogen source, a carbon source, various growth factors required by the organism, and water. Stationary phase cultures are typically preferred as maximum cell yield is obtained. Several Lactobacillus strains have also demonstrated highest resistance to drying when harvested in the stationary phase (B. M. Corcoran et al., J. Appl. Microbiol. 2004, 96, 1024-1039; and G. Broeckx et al., Dry. Technol. 2020, 38, 1474-1492).

Preparation of Probiotics Cell Suspension

Single colonies of LGG (U.S. Pat. No. 4,839,281), L. plantarum and L. paracasei were inoculated in sterile MRS broths and incubated aerobically at 37° C. for 24 h (for LGG and L. plantarum) and at 30° C. for 72 h (for L. paracasei), wherein a stationary phase cell concentration of approximately 10⁸-10⁹ colony forming units/mL (CFU/mL) was attained for each bacterium. The probiotic cells were then washed thrice with 0.9% (w/v) NaCl, with centrifugation at 10,000×g for 5 min between each wash. Next, the probiotic cell pellets were resuspended in 0.9% NaCl at one-tenth of the initial inoculated volume to obtain a 10× concentrated probiotic sample. Ten-fold serial dilutions and drop plating on MRS agar were performed to determine the initial CFU concentration.

### Example 1. Spray-Drying Encapsulation of LGG

Calcium Chloride-Alginate-Sucrose Spray-Drying Encapsulation of LGG

The LFR5/60 alginate (M/G=30/70, Mw=20-60 kDa), was the polymer of choice and the disaccharide, sucrose, was used as the protective agent. 2.22% (w/v) alginate with 11.11% (w/v) of sucrose were dissolved in distilled water and sterilized by autoclaving at 121° C. for 15 minutes. 10 mM CaCl₂ was used as the crosslinking agent and it was prepared by following the preparation method for alginate and sucrose. 10 mM CaCl₂ was chosen as higher concentrations of calcium chloride were observed to cause frequent spray-drier nozzle blockage. 10× concentrated LGG cell suspension was then added in a 1:9 (v:v) ratio to the alginate-sucrose polymeric slurry, thereby yielding a final 2% (w/v) alginate, 10% (w/v) sucrose with approximately 3×10⁸ CFU/mL of LGG.

The Buchi-290 spray-dryer (Buchi AG, Switzerland) was set up with assembly of the Buchi three-fluid co-axial nozzle. Nitrogen gas flow was set at 742 L/hr and the spray-dryer equipment was pre-heated at 140° C. to sterilize and dry-out any residual moisture in the apparatus. Next, the inlet temperature of the spray-dryer was set to 120° C., and the alginate-sucrose-LGG slurry and CaCl₂ crosslinking agent were respectively fed into the inner and outer feed channels. 1.5 mL/min inner channel flow rate and flow rate ratio (inner:outer) of 1:2 was used. Batch volumes of 25-50 mL of alginate-sucrose-LGG slurry were prepared at one go, and the outlet temperatures ranged from 62-77° C. Following spray-drying, the crosslinked spray-dried alginate-sucrose-LGG powder was collected at the collecting chamber and evaluated on various accounts as detailed accordingly in the following examples.

Control Sets

The four control sets are control A—10% (w/v) sucrose (SUC), control B—2% alginate (ALG), control C—2% alginate with 10% sucrose (ALG-SUC), and control D—2% alginate with 10 mM CaCl₂ crosslinking (Ca-ALG). The four control sets were prepared from their respective starting materials by following the spray-drying protocol above.

Characterization

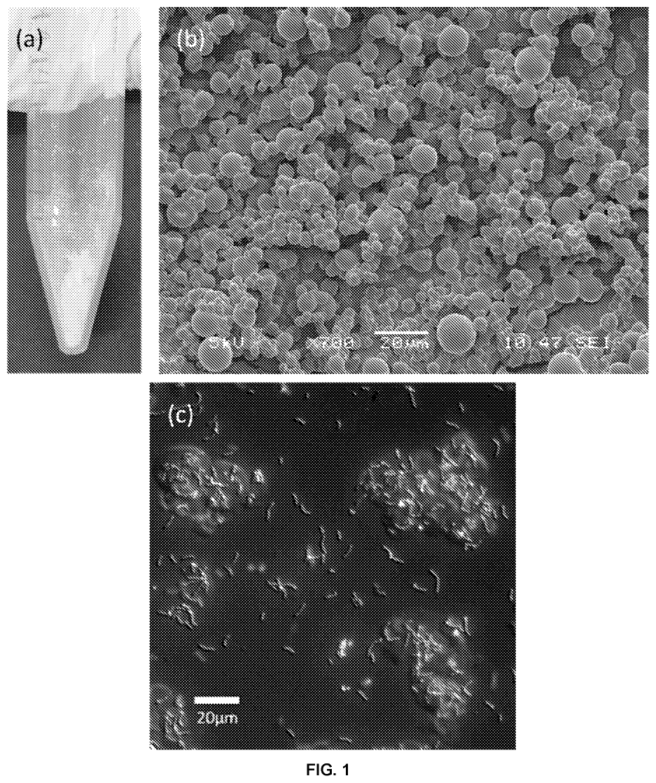

A fine, homogenous and off-white powder was yielded for the Ca-ALG-SUC formulation (FIG. 1a). SEM showed a powder particle size of 2-15 μm for the Ca-ALG-SUC sample (FIG. 1b). Upon rehydration in sterile 0.9% (w/v) NaCl solution, viable LGG bacteria could be seen embedded in calcium-alginate gels, indicating that effective crosslinking was achieved (FIG. 1c).

### Example 2. Survivability of LGG in Various Formulations Post-Spray-Drying

The survivability of LGG in the various formulations post-spray-drying in Example 1 was evaluated.

Determination of Viable LGG Cell Quantity

The viable LGG cell quantity present in the various formulations was determined by dissolving approximately 50 mg of spray-dried LGG powder (SUC, ALG, ALG-SUC, Ca-ALG or Ca-ALG-SUC) in 5 mL of 50 mM sodium citrate. The powder was thoroughly dissolved by vortexing, then serial dilution and drop plating on MRS agar were performed to determine CFU counts.

Results and Discussion

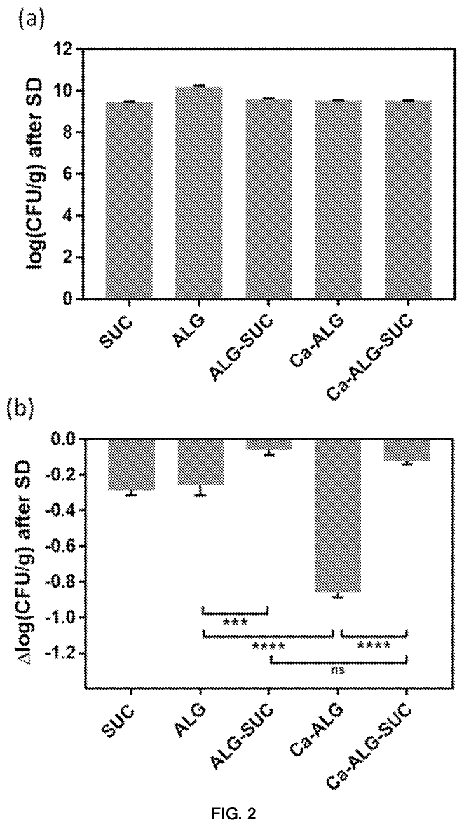

As seen in FIG. 2, a high LGG cell density of approximately 3.5 billion CFU/g or 9.5 log(CFU/g) was attained, indicating that less than 0.15 log(CFU/g) was lost following spray-drying. The addition of sucrose was also found to significantly improve LGG survivability following spray-drying, likely due to its stabilizing effect on LGG cellular membranes and proteins. Comparing crosslinked and un-crosslinked samples (ALG vs Ca-ALG and ALG-SUC vs Ca-ALG-SUC), calcium crosslinking greatly reduced LGG survivability in ALG samples, but did not have a significant impact on LGG survivability in ALG-SUC. A similar phenomenon has been observed in freeze-dried crosslinked alginate, where divalent cations, including Ca²⁴, demonstrated antagonistic properties in reducing desiccation survivability of probiotics (L. L. Tan et al., Food Bioprod. Process. 2020, 124, 369-377). In the same study (L. L. Tan et al., Food Bioprod. Process. 2020, 124, 369-377), sucrose was found to reverse the deleterious effects of divalent cations and restore viability of probiotics in freeze-dried calcium alginate particles. Possibly, a similar mechanism of Ca²⁺ antagonism towards LGG survivability occurred in this spray-drying process, and the addition of sucrose as a protective agent alleviated this effect.

### Example 3. SGF and SIF Exposure Tests

The crosslinked spray-dried Ca-ALG-SUC with LGG formulation, and control set LGG samples (SUC, ALG, ALG-SUC, Ca-ALG) prepared in Example 1 were also subjected to SGF and SIF exposure tests. The encapsulated probiotic powders (SUC, ALG, ALG-SUC, Ca-ALG or Ca-ALG-SUC) were exposed to SGF to determine their ability to maintain LGG viability in the human gastric environment.

SGF Exposure Tests

SGF was prepared by adjusting the pH of 0.2 M NaCl solution to pH 2 with HCl, with addition of 2000 units/mL of porcine pepsin. 50±2 mg of spray-dried LGG powders (ALG, ALG-SUC, Ca-ALG or Ca-ALG-SUC) were added to 5 mL of SGF. Other tested powder:SGF ratios include 25±1 mg:5 mL and 15±0.6 mg:7.5 mL. SGF adjusted to pH 2.5 and pH 3 were also tested for the SUC samples to determine the intrinsic acid resistance of LGG, without any addition of the crosslinking alginate polymer. Samples were incubated at 37° C., 200 rpm shaking conditions for 2 hours. Next, the SGF mixture was pelleted down by centrifugation at 10,000×g for 5 min, and the SGF was replaced with 5 mL of 50 mM sodium citrate. Samples were thoroughly dissolved in sodium citrate by vortexing, then viable probiotic counts were determined by serial dilution and drop-plating. Viable probiotic counts after SGF exposure were determined by dissolution in 0.05 M sodium citrate and drop-plating on MRS agars.

SIF Exposure Tests

SIF was prepared with 200 U/mL α-amylase from porcine pancreas in PBS (pH 7.4). SIF exposure tests were performed by following the protocol above except SIF was used instead of SGF, and the powder:SIF ratio was 50±2 mg:5 mL.

Results and Discussion

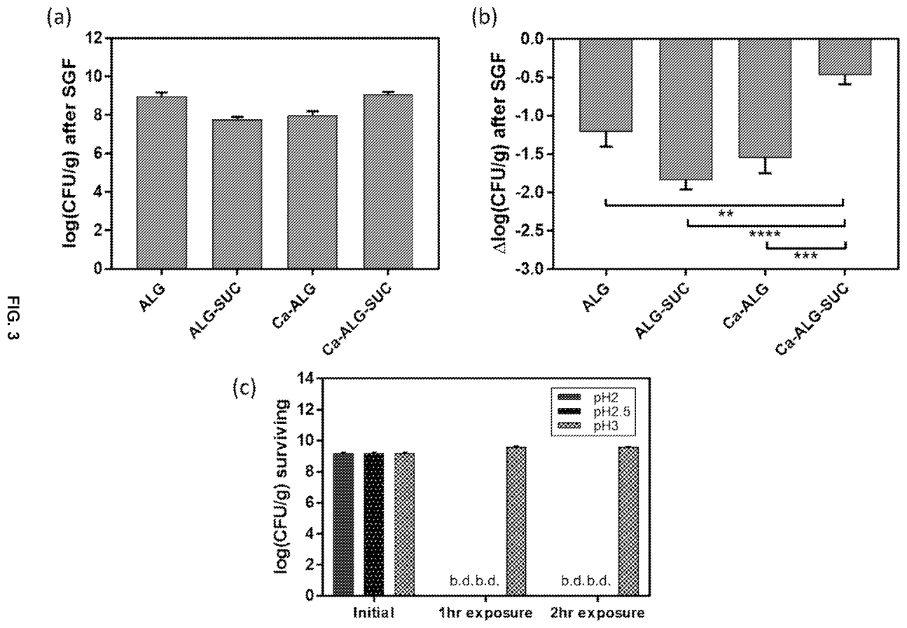

As seen in FIG. 3, LGG survival in SGF was greatly enhanced in the Ca-ALG-SUC formulation, as compared to unencapsulated LGG in the SUC formulation. The Ca-ALG-SUC formulation showed highest viability after SGF exposure, with approximately 0.5 log(CFU/g) reduction, as compared to the control sets.

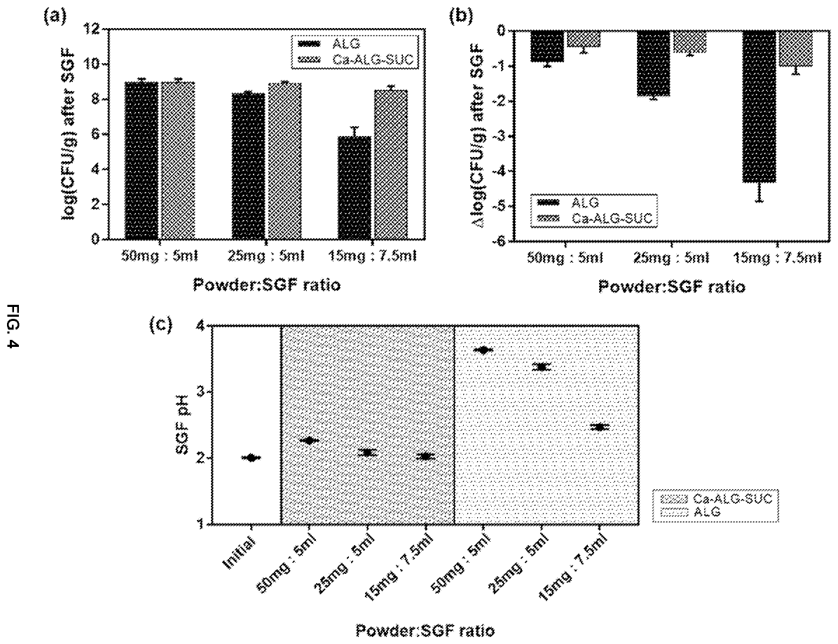

A hypothesis for this enhanced microorganism survivability in the crosslinked spray-dried alginate matrix, is due to a localized pH buffering effect achieved within each spray-dried particle. By the conversion of alginate to alginic acid, excess protons in the vicinity of embedded LGG are sequestered, thereby maintaining a localized pH higher than the external SGF milieu. This hypothesis was tested by exposing the un-crosslinked ALG and crosslinked Ca-ALG-SUC formulations to SGF, in varying powder mass to SGF volume ratios (FIG. 4). As seen in FIG. 4a-b, high log(CFU/g) counts were obtained for the crosslinked Ca-ALG-SUC formulation, even with decreasing powder mass to SGF volume ratio. In contrast, survivability of LGG in the un-crosslinked ALG formulation was significantly decreased with decreasing powder mass to SGF volume ratio. The final pH value of the SGF milieu following addition of formulation powder was also measured (FIG. 4c), and the pH value of the SGF consisting crosslinked Ca-ALG-SUC formulation was relatively constant, unlike for ALG, where pH was significantly decreased with decreasing powder mass to SGF volume ratio. In acidic pH, alginate is known to sequester protons and convert to insoluble alginic acid, thereby achieving a pH buffering effect which promotes survivability of encapsulated probiotics (K. Lee & D. Mooney, LWT. 2012, 72, 390-398). Data from FIG. 4 suggest that the un-crosslinked ALG formulation conferred gastroprotection to encapsulated LGG primarily through buffering pH of the entire gastric fluid environment. Accordingly, as the powder mass to SGF volume decreased, the pH buffering capacity of un-crosslinked ALG was hampered, thereby exposing LGG to the detrimental acidic pH, causing significant viability losses. The pH buffering capacity of crosslinked Ca-ALG-SUC was however, independent of the SGF volume, likely due to a localized pH buffering effect achieved within each spray-dried particle, facilitated by the formation of crosslinked calcium alginate gels surrounding each particle. The crosslinked calcium alginate surface layer likely immobilized encapsulated LGG probiotics within the matrix. By the conversion of alginate to alginic acid, excess protons in the vicinity of embedded LGG were sequestered, thereby maintaining a localized pH higher than the external SGF milieu, and maintaining high LGG viability. Overall, the pH buffering capacity of the crosslinked Ca-ALG-SUC formulation is independent of the SGF volume administered, and this provides more versatility in its application to various hosts.

### Example 4. Storage Stability of Ca-ALG-SUC LGG

The storage stability of Ca-ALG-SUC with LGG (prepared in Example 1) over a period of 4 and 8 weeks was tested.

Storage Stability Test

The spray-dried sample (Ca-ALG-SUC) was stored at 4° C. refrigerated conditions over a 4- and 8-week period. To maintain a dry environment, MiniPax absorbent packets (Sigma-Aldrich, USA) were placed as desiccants together with the spray-dried powders. Following storage, viable LGG counts were determined by dissolution in 50 mM sodium citrate and drop-plating on MRS agars. The sample (Ca-ALG-SUC) was also exposed to SGF pH 2 after storage by following the method described in Example 3, to determine if gastroprotective effect of the formulation was retained after storage.

Results and Discussion

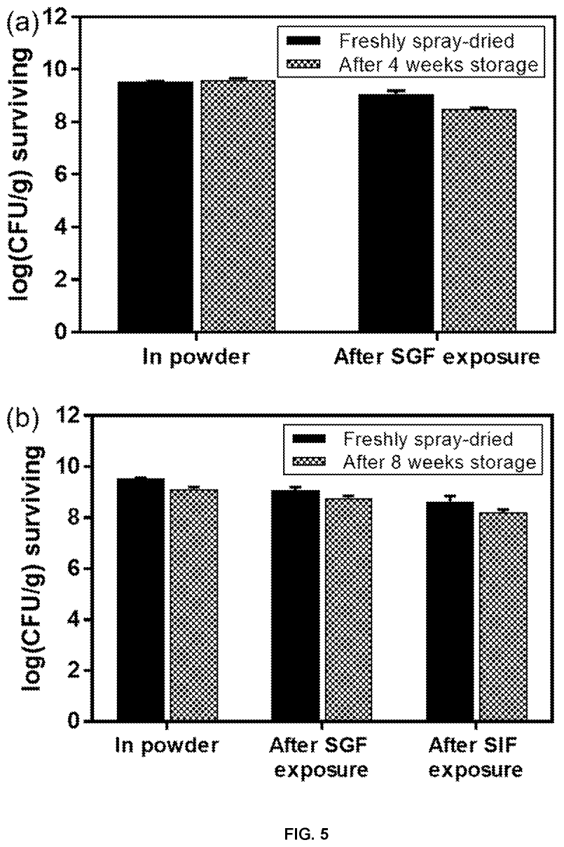

As seen in FIG. 5, high log(CFU/g) counts were obtained following 4 weeks (FIG. 5(a)) and 8 weeks (FIG. 5(b)) of refrigerated storage, and the gastroprotective function of the Ca-ALG-SUC matrix was also retained.

Overall, the various data, including microorganism survivability post-spray-drying, microorganism survivability following SGF/SIF insult, and storage stability, demonstrate that the Ca-ALG-SUC formulation was effective in producing and in encapsulating microorganisms for gastrointestinal delivery.

### Example 5. Calcium Chloride-Alginate-Sucrose Spray-Drying Encapsulation of LGG with Feed Channels Inverted

In this example, the same alginate type, alginate concentration, sucrose concentration, calcium chloride concentration, microorganism and spray-drying parameters were used as in Example 1, except that the CaCl₂ crosslinking agent and the polymeric-microorganism slurry were switched in the feed positions. 10 mM CaCl₂ was fed to the inner channel of the spray-drying nozzle, while the alginate-sucrose-LGG mixture was fed to the outer channel. The survivability of LGG was determined by following the protocol in Example 2 and the SGF exposure test was performed by following the protocol in Example 3.

Results and Discussion

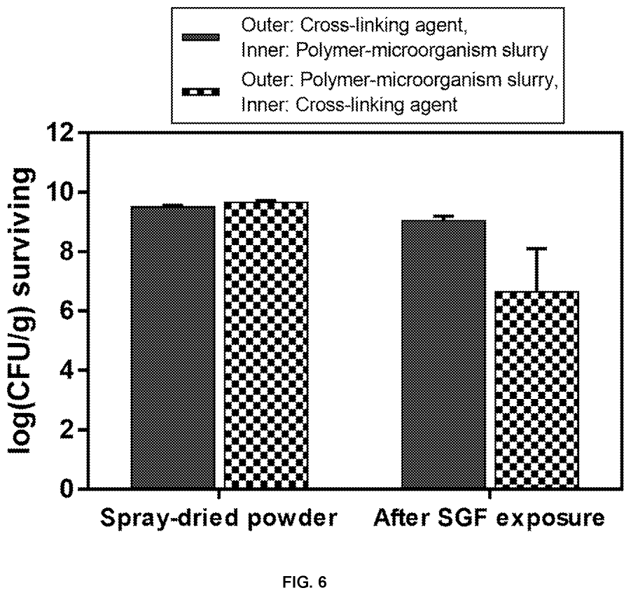

As shown in FIG. 6, high log(CFU/g) counts were obtained for both Ca-ALG-SUC formulations. The difference between the two formulations lies in the LGG survivability after SGF exposure, where the formulation with crosslinking agent in the inner feed channel performed significantly worse. This suggests that an alternative mechanism for the gastroprotective effect of Ca-ALG-SUC (with crosslinking agent on the outer channel) on encapsulated microorganisms, is by the formation of a crosslinked calcium-alginate layer surrounding each particle, which immobilizes the microorganisms within each particle, providing them with a localized pH buffering effect.

### Example 6. Calcium Chloride-Alginate Spray-Drying Encapsulation of Lactiplantibacillus Plantarum and Lacticaseibacillus Paracasei

In this example, similar SUC and Ca-ALG-SUC formulations as described in Example 1 were used, except that the microorganism was changed to either L. plantarum or L. paracasei (own strain isolated from water kefir). The post-spray-drying survivability of L. plantarum or L. paracasei was determined by following the protocol in Example 2 and the SGF exposure test was performed by following the protocol in Example 3.

Results and Discussion

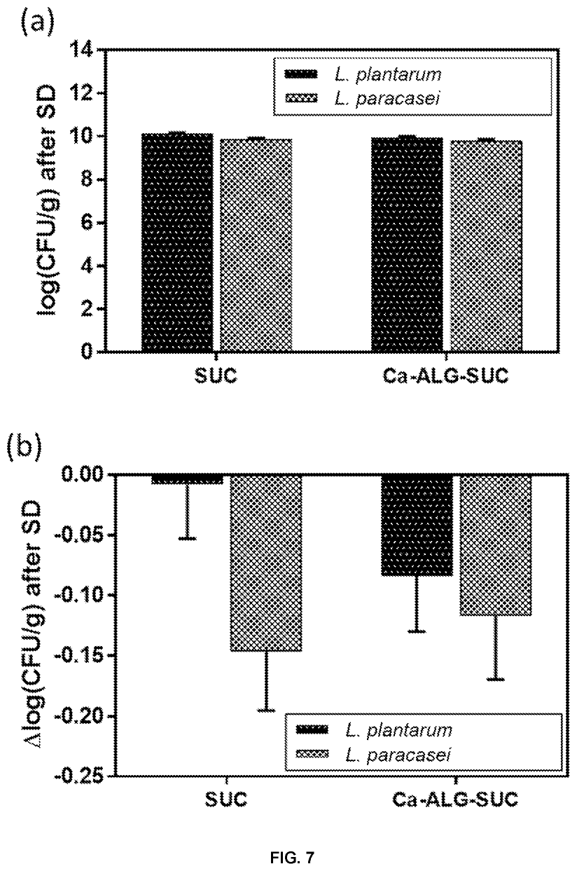

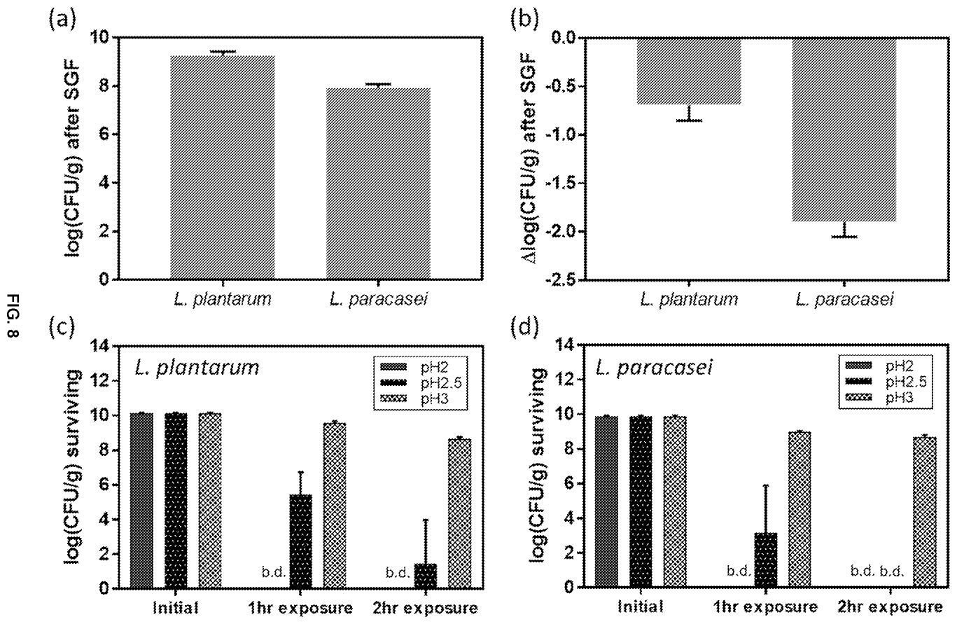

FIG. 7 shows high survival of both lactobacilli strains in the Ca-ALG-SUC formulation, with approximately 10 log(CFU/g) attained post-spray-drying for each strain, indicating that less than 0.15 log(CFU/g) was lost during spray-drying. FIG. 8 demonstrates a gastroprotective function by the Ca-ALG-SUC matrix on both lactobacilli strains, where high log(CFU/g) of both strains were retained following SGF exposure.

### Example 7. Calcium-Alginate Spray-Drying Encapsulation with Other Protective Agents

Other protective agents, namely trehalose, cysteine and sodium ascorbate, were incorporated in the calcium-alginate spray-drying formulation to seek improved room temperature shelf-life properties for LGG probiotics. The calcium chloride crosslinking solution was also replaced with 10 mM calcium lactate, as calcium lactate is known to have a neutral taste, hence better organoleptic properties. Three formulations were compared in this example, namely: 1) 5% (w/v) sucrose and 5% trehalose (Ca-Alg-Suc-Tre); 2) 5% sucrose, 5% trehalose and 0.2% L-cysteine (Ca-Alg-Suc-Tre-Cys); and 3) 5% sucrose, 5% trehalose and 0.2% sodium ascorbate (Ca-Alg-Suc-Tre-Asc). These formulations were prepared and their conditions were assessed by following the protocols in Examples 1-4.

Results and Discussion

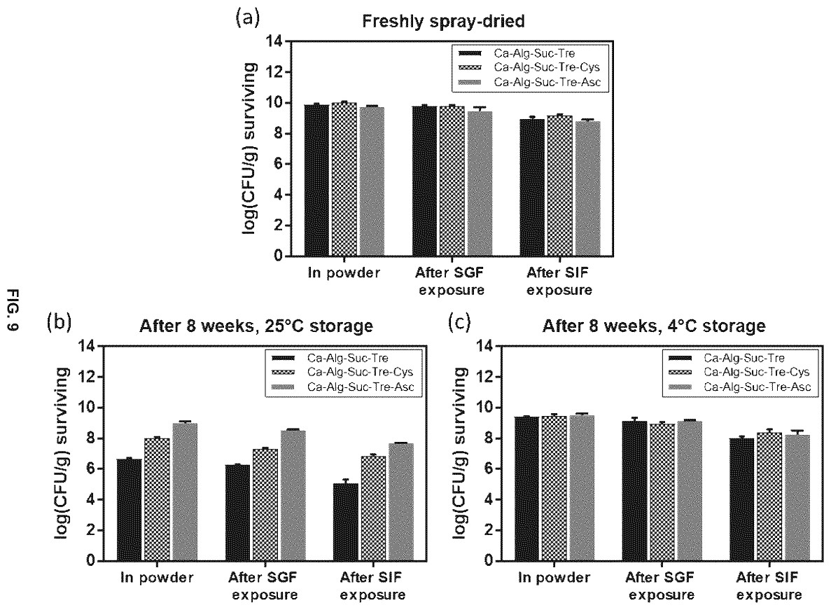

From FIG. 9, all three tested formulations showed high survival of LGG probiotics post spray-drying and post-SGF/SIF exposure, indicating that a change of protective agents did not affect the calcium alginate crosslinking process. High CFU counts of LGG were retained following 8 weeks of refrigerated storage, and the Ca-Alg-Suc-Tre-Asc formulation showed highest LGG survival for 8 weeks of room temperature storage. These results suggest the efficacy of adding antioxidant agents, like ascorbate, in improving shelf-life properties of the spray-dried microorganism formulation.

1. A method of forming polymeric microparticles housing live microorganisms, the method comprising the steps of:
(a) providing:
a microbial solution comprising
a crosslinkable polymeric material compatible with live microorganisms;
a protective agent;
a microbial population; and
water; and
a crosslinking agent solution comprising a crosslinking agent and water; and
(b) subjecting the microbial and crosslinking agent solutions to spray-drying using a co-axial nozzle configured to spray at least three fluids independently, thereby producing microparticles of a crosslinked polymeric material housing live microorganisms, wherein:
the microbial solution is sprayed as a first feed through an inner channel, the crosslinking agent solution is sprayed as a second feed through an outer channel that surrounds the inner channel; and an atomizing gas is sprayed through a jacketing channel that surrounds the outer channel; or
the crosslinking agent solution is sprayed as a first feed through an inner channel, the microbial solution is sprayed as a second feed through an outer channel that surrounds the inner channel; and an atomizing gas is sprayed through a jacketing channel that surrounds the outer channel.

2. The method according to claim 1, wherein the crosslinkable polymeric material compatible with live microorganisms is selected from one or more of the group consisting of an oligosaccharide, dextrin, or more particularly, alginate, a gum (e.g. Arabic, guar, locust bean, xanthan, glucomannan), a non-bioactive protein (e.g. gelatin, collagen, whey protein, soy protein, or caseinate), carrageenan, pectin, cellulose, and starch.

3. The method according to claim 2, wherein the crosslinkable polymeric material compatible with live microorganisms is alginate.

4. The method according to claim 1, wherein the crosslinkable polymeric material compatible with live microorganisms has a concentration of from 2 to 10% w/v.

5. The method according to claim 4, wherein the crosslinkable polymeric material compatible with live microorganisms has a viscosity at room temperature of from 5 mPas to 250 mPas (measured at a shear rate of 100/s).

6. The method according to claim 1, wherein the protective agent is selected from one or more of the group consisting of a sugar (e.g. a monosaccharide, a disaccharide), an amino acid, a protein, a prebiotic, and an antioxidant.

7. (canceled)

8. The method according to claim 1, wherein the protective agent has a concentration of from 5 to 30% w/v in the microbial solution.

9. The method according to claim 1, wherein the dry weight percentage of the crosslinkable polymeric material compatible with live microorganisms is from 10 to 90% of the total dry weight.

10. The method according to claim 1, wherein the microbial population is selected from one or more of probiotic bacteria and/or a live biotherapeutic.

11. The method according to claim 10, wherein the microbial population is formed from one or more of the group consisting of Lacticaseibacillus rhamnosus, Lacticaseibacillus paracasei, and Lactiplantibacillus plantarum.

12. (canceled)

13. The method according to claim 1, wherein the crosslinking agent is selected from one or more of the group consisting of an inorganic salt of a multivalent cation, a polyanion, and genipin.

14. (canceled)

15. The method according to claim 13, wherein the crosslinking agent is calcium chloride.

16. The method according to claim 1, wherein the concentration of the crosslinking agent solution is from 1 to 100 mM.

17. The method according to claim 1, wherein an inlet temperature of a drying gas used in the method is greater than or equal to 100° C.

18. (canceled)

19. The method according to claim 1, wherein:
when the first feed is the microbial solution and the second feed is the crosslinking agent solution, then the ratio of the first feed flow rate through the inner channel to the second feed flow rate through the outer channel is from 1:1 to 0.1:1; or
when the first feed is the crosslinking agent solution and the second feed is the microbial solution, then the ratio of the first feed flow rate through the inner channel to the second feed flow rate through the outer channel is from 10:1 to 1:1.

20. (canceled)

21. (canceled)

22. The method according to claim 1, wherein the co-axial nozzle is operated in one or both of:
(ai) a pneumatic atomization mode; and
(aii) a co-current mode.

23. The method according to claim 1, wherein the live microorganisms in the microparticles of the crosslinked polymeric material housing live microorganisms remain alive for a period of from 12 to 18 months from the formation of said microparticles.

24. (canceled)

25. The method according to claim 1, wherein the microbial solution further comprises a non-microbial bioactive.

26. The method according to claim 25, wherein the non-microbial bioactive is selected from one or more of the group consisting of nutraceuticals (e.g. phytochemicals), lipids, and bioactive proteins.

27. (canceled)

28. The method according to claim 1, wherein the crosslinking agent solution further comprises chitosan, so as to provide chitosan-coated microparticles.
